(19) United States
(12) Patent Application Publication
(10) Pub. No.: US 2020/0022754 A1
(43) Pub. Date: Jan. 23, 2020

(54) SYSTEMS AND METHODS FOR TISSUE DISPLACEMENT

(71) Applicant: Robert J. COTTONE, Davie, FL (US)

(72) Inventor: Robert J. COTTONE, Davie, FL (US)

(21) Appl. No.: 16/576,976

(22) Filed: Sep. 20, 2019

Related U.S. Application Data
(63) Continuation of application No. 16/495,660.
(60) Provisional application No. 62/476,312, filed on Mar. 24, 2017.
(60) Provisional application No. 62/505,475, filed on May 12, 2017.
(60) Provisional application No. 62/560,725, filed on Sep. 20, 2017.

(51) Int. Cl. A61B 18/14; A61B 5/06

(52) U.S. Cl. CPC A61B 18/1492; A61B 2018/00577; A61B 5/061

(57) ABSTRACT

A medical device including a handle; a flexible conduit having a proximal segment and a distal segment, wherein the proximal segment is coupled to the handle; and a substantially contiguous shaping structure coupled to the distal segment of the flexible conduit, wherein the shaping structure is configured to transition from (i) a substantially linear configuration to (ii) a configuration where a portion of the contiguous shaping structure is laterally displaced from remaining portions of the contiguous support structure upon the application of an axial compression force to the shaping structure.

### CROSS-REFERENCE TO RELATED APPLICATION

This application is a continuation of application Ser. No. 16/495,660, filed Sep. 19, 2019, entitled SYSTEMS AND METHODS FOR TISSUE DISPLACEMENT, which application is a national stage application of International Application Ser. No. PCT/US2018/024334, filed Mar. 26, 2018, entitled SYSTEMS AND METHODS FOR TISSUE DISPLACEMENT, which application is related to and claims priority to each of U.S. Provisional Patent Application Ser. No. 62/476,312, filed Mar. 24, 2017; U.S. Provisional Patent Application Ser. No. 62/505,475, filed May 12, 2017; and U.S. Provisional Patent Application Ser. No. 62/560,725, filed Sep. 20, 2017, the entirety of all of which is incorporated herein by reference.

### STATEMENT REGARDING FEDERALLY SPONSORED RESEARCH OR DEVELOPMENT

n/a

### FIELD OF THE INVENTION

The present disclosure relates to devices, systems, and methods of use thereof for displacing and/or manipulating anatomical structures and tissue for treatment.

### BACKGROUND OF THE INVENTION

Various medical procedures involve the application or delivery of energy and/or radiation to targeted areas of the body. For example, thermal and radiofrequency energy can be delivered (or removed, in the case of cooling) to ablate problematic tissue regions and/or to interrupt a natural physiological response or process (such as inflammation). Radiation is often used to target and destroy cancerous growths in various parts of the body. During such treatments, there may be a risk of inadvertent or undesirable exposure of non-targeted tissue to such energies/treatments, and thus, resulting complications for otherwise healthy tissue.

For example, various modalities such as radiofrequency and cryogenic ablation are employed to treat atrial fibrillation and other cardiac arrhythmias. During ablation, there is a risk of thermal damage to the esophagus due to its proximity and contact with the left atrium, which increase the risks of the formation of an atrio-esophageal fistula. Patients with this complication have close to an 80% mortality rate from stroke, mediastinitis, sepsis, and endocarditis. Chavez et al. “Atrioesophageal Fistula following Ablation Procedures for Atrial Fibrillation: Systematic Review of Case Reports.” Open Heart 2.1 (2015): 1-8. Even without formation of a fistula, there exists a continuum of damage to the esophagus from such ablation techniques ranging from superficial thermal injury to necrosis or ulcer. Nair et al. “Atrioesophageal Fistula: A Review.” Journal of Atrial Fibrillation 8.3 (2015): 1331. Pappone et al. “Atrio-Esophageal Fistula After AF Ablation: Pathophysiology, Prevention & Treatment.” Journal of Atrial Fibrillation 6.3 (2013): 860.

In another example, radiation treatments may be used to target tumors that are in close proximity to non-targeted vital organs, such as the heart when dealing with breast cancer, and the rectum, bladder and/or urethra when dealing with prostate cancer. Such treatments would benefit from improved minimally-invasive approaches to displacing or otherwise shifting the position of such healthy tissue structures and organs away from the targeted treatment areas to reduce the likelihood of collateral tissue damage and associated complications.

### SUMMARY OF THE INVENTION

The present disclosure provides a medical device, including a handle; a flexible conduit having a proximal segment and a distal segment, wherein the proximal segment is coupled to the handle; and a substantially contiguous shaping structure coupled to the distal segment of the flexible conduit, wherein the shaping structure is configured to transition from (i) a substantially linear configuration to (ii) a configuration where a portion of the shaping structure is laterally displaced from remaining portions of the contiguous shaping structure upon the application of an axial compression force to the shaping structure. The shaping structure may extend along a substantial length of the medical device. The portion of the contiguous shaping structure may be laterally displaced substantially within a single plane. The shaping structure may include a unitary spine defining a plurality of radially offset living hinges. The shaping structure may include a first plurality of living hinges; a second plurality of living hinges radially offset from the first plurality of living hinges between approximately 150 degrees and approximately 210 degrees; a third plurality of living hinges substantially radially aligned with the second plurality of living hinges; and a fourth plurality of living hinges substantially radially aligned with the first plurality of living hinges.

The shaping structure may include a segment between the second plurality of living hinges and the third plurality of living hinges that substantially resists bending from the application of the axial compression force. The segment may include a plurality of living hinges extending along a longitudinal length of the segment, wherein each living hinge of the plurality is angularly offset by approximately 180 degrees with respect to a consecutive living hinge of the plurality, and a plurality of stopping elements, wherein each stopping element is radially offset by each living hinge of the plurality by approximately 180 degrees to restrict a motion range of the respective living hinge.

The first plurality of living hinges may provide at least one of a turn and an arc of approximately 90 degrees from the application of the axial compression force. The second plurality of living hinges may provide at least one of a turn and an arc of approximately 90 degrees from the application of the axial compression force. Each of the third and fourth pluralities of living hinges may provide at least one of a turn and an arc of approximately 90 degrees from the application of the axial compression force.

The medical device may include a pull wire coupled to the handle and the shaping structure, wherein the pull wire is configured to apply an axial compression force to at least a portion of the shaping structure. The shaping structure may define a lumen therethrough, the lumen defining an oblong cross-sectional opening, and the pull wire may traverse the lumen.

The flexible conduit may be configured to substantially resist axial compression and/or include at least one of a stainless steel hypotube and a nitinol hypotube.

The medical device may include a plurality of balloons coupled to the shaping structure. Each of the balloons may be longitudinally spaced along a length of the shaping structure, and at least one of the balloons may be non-concentric with the shaping structure. At least one of the balloons may be expandable asymmetrically about a circumference of the shaping structure. At least one of the balloons may have a substantially semi-circular cross-section when inflated. At least one of the balloons may have a substantially flattened surface segment when inflated. At least one of the balloons may be radially offset with respect to at least one other balloon. At least one of the balloons may be radially offset with respect to at least one other balloon between approximately 150 degrees and approximately 210 degrees. Each of the balloons of the plurality of balloons may be individually inflatable.

The flexible conduit may include a plurality of living hinges, wherein each living hinge is angularly offset between approximately 70 degrees and 110 degrees with respect to the nearest living hinge of the plurality.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is an illustration of an example of a tissue displacement device constructed in accordance with the principles of the present disclosure;

FIG. 2 is an illustration of an example of a proximal segment of a tissue displacement device constructed in accordance with the principles of the present disclosure;

FIG. 3 is an illustration of an alternative configuration of the proximal segment of FIG. 2;

FIG. 4 is a cross sectional illustration of a segment of the proximal segment of FIG. 2;

FIG. 5 is an illustration of an example of a geometric configuration of a tissue displacement device constructed in accordance with the principles of the present disclosure;

FIG. 6 is an illustration of an alternative configuration of the tissue displacement device of FIG. 1 with one or more outer layers removed for the sake of illustration certain features;

FIG. 7 is another illustration of select components of the tissue displacement device of FIG. 1;

FIG. 8 is yet another illustration of select components of the tissue displacement device of FIG. 1;

FIG. 9 is an illustration of an example of a shaping structure of a tissue displacement device constructed in accordance with the principles of the present disclosure;

FIG. 10 is an illustration of an example of an interlocking joint of a shaping structure of a tissue displacement device constructed in accordance with the principles of the present disclosure;

FIG. 11 is an illustration of an example of a segment of a shaping structure of a tissue displacement device constructed in accordance with the principles of the present disclosure;

FIG. 12 is an additional illustration of the segment shown in FIG. 11;

FIG. 13 is an illustration of another example of a segment of a shaping structure of a tissue displacement device constructed in accordance with the principles of the present disclosure;

FIG. 14 is an illustration of variable characteristics of living hinges for a shaping structure of a tissue displacement device constructed in accordance with the principles of the present disclosure;

FIG. 15 is an illustration of an example of a living hinge geometry for a shaping structure of a tissue displacement device constructed in accordance with the principles of the present disclosure;

FIG. 16 is an illustration of another example of a living hinge geometry for a shaping structure of a tissue displacement device constructed in accordance with the principles of the present disclosure;

FIG. 17 is an illustration of additional examples of living hinge geometries for a shaping structure of a tissue displacement device constructed in accordance with the principles of the present disclosure;

FIG. 18 is an illustration of additional examples of living hinge geometries for a shaping structure of a tissue displacement device constructed in accordance with the principles of the present disclosure;

FIG. 19 is an illustration of an example of a shaping structure in a multi-planar configuration;

FIG. 20 is an additional view of the shaping structure of FIG. 19;

FIG. 21 is an additional view of the shaping structure of FIG. 19;

FIG. 22 is an illustration of an example of an articulating segment of a shaping structure of a tissue displacement device constructed in accordance with the principles of the present disclosure;

FIG. 23 is an additional view of the articulating segment of FIG. 22;

FIG. 24 is an illustration of another example of an articulating segment of a shaping structure of a tissue displacement device constructed in accordance with the principles of the present disclosure;

FIG. 25 is an additional view of the articulating segment of FIG. 24;

FIG. 26 is an illustration of an example of a shaping structure of a tissue displacement device constructed in accordance with the principles of the present disclosure;

FIG. 27 is a cross-sectional illustration of an example of a balloon of a tissue displacement device constructed in accordance with the principles of the present disclosure;

FIGS. 28A, 28B, 28C and 28D are cross-sectional illustrations of another example of a balloon of a tissue displacement device constructed in accordance with the principles of the present disclosure;

FIG. 28E is an illustration of a segmented balloon.

FIG. 28F is a cross-sectional illustration of FIG. 28E.

FIG. 29 is an illustration of an example of a handle and pull wire configuration for a tissue displacement device constructed in accordance with the principles of the present disclosure;

FIG. 30 is an alternative position of the handle and pull wire of FIG. 29;

FIG. 31 is an illustration of an example of a cross-section of a lumen for a tissue displacement device constructed in accordance with the principles of the present disclosure;

FIG. 32 is an illustration of another example of a lumen for a tissue displacement device constructed in accordance with the principles of the present disclosure;

FIG. 33 is an illustration of an alternative configuration of the segment of the device shown in FIG. 32;

FIG. 34 is an illustration of yet another example of a cross-section of a lumen for a tissue displacement device constructed in accordance with the principles of the present disclosure;

FIGS. 35A-D are illustrations of additional examples of cross-sections of lumens for a tissue displacement device constructed in accordance with the principles of the present disclosure;

FIG. 36 is an illustration of an example of a distal segment of a tissue displacement device constructed in accordance with the principles of the present disclosure;

FIG. 37 is an illustration of an exemplary use of a tissue displacement device in an esophagus in accordance with the principles of the present disclosure;

FIG. 38 is another illustration of an exemplary use of a tissue displacement device in an esophagus in accordance with the principles of the present disclosure; and

FIG. 39 is an illustration of an exemplary use of a tissue displacement device in a gastric region in accordance with the principles of the present disclosure.

### DETAILED DESCRIPTION OF THE INVENTION

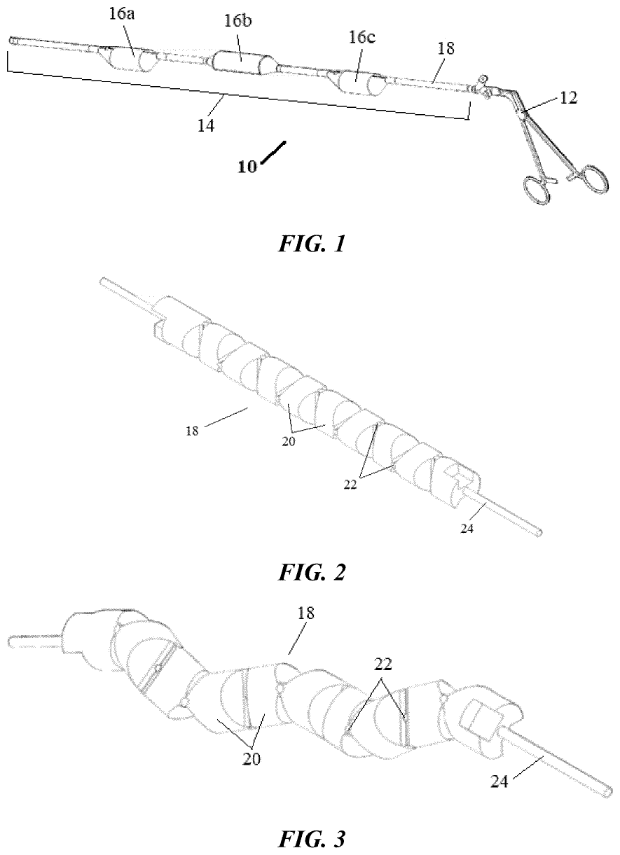

The present disclosure provides systems, devices, and methods thereof for minimally-invasive approaches, endoscopically or laparoscopically, to accessing, displacing or otherwise shifting the position of healthy tissue structures and organs away from disruptive or harmful targeted treatment areas to reduce the likelihood of collateral tissue damage and associated complications. Now referring to the figures, an example of a tissue displacement device 10 is shown. As shown in FIG. 1, the device 10 generally includes a handle 12 and an elongate body 14 sized for use in and around various anatomical structures, e.g., esophagus, trachea, stomach, colon, vasculature (arterial and venous), orifices, or other body cavities (e.g., the peritoneum) to facilitate tissue displacement as described herein. The device described herein may be scaled and dimensioned for use intravascularly, intraluminally, percutaneously, transdermally, laparoscopically, or otherwise. The elongate body 14 may be selectively adjustable and/or operable through manipulation of the handle 12 to take on one or more geometric configurations suitable for a particular treatment or procedures. The device 10 may include one or more expandable elements, such as balloons 16a, 16b, 16c (collectively, ‘16’) that can be positioned at one or more locations along the length of the elongate body 14 to facilitate contact and/or force exertion or dispersion of contact force against a particular tissue site, as described further herein.

Continuing to refer to FIG. 1, the elongate body 14 may include a flexible conduit 18 having a proximal segment coupled to the handle 12, and a distal segment opposite the proximal segment. The flexible conduit 18 is flexible in one or more planes, has a selectable degree of resistance to axial compression and provides a high degree of torque transmission whether the conduit 18 is in a substantially linear configuration (such as that shown in FIG. 1) or in a multi-planar, contoured configuration. The flexible conduit 18 may be a hypotube with one or more cut patterns through the wall of the hypotube along the length.

The conduit 18 may define one or more lumens or passages therethrough for the passage of one or more pull wires, device control elements, electrical wires or conduits, fluid lumens or passages, and the like. In one example, the conduit 18 may include a hypotube, a compressed coil, a polymer tube, or a polymer tube incorporating a braid or coil within the tubular wall or other similar component(s). There may be one or more flexible conduits arranged together in-line (axially) where one flexible conduit is fixed to an adjacent conduit. The flexible conduit may be constructed from stainless steel, nitinol, polymers, carbon fiber and/or combinations and composites thereof. Examples of materials that may be used include stainless steel (SST), Nitinol, or polymers. Examples of other metals which may be used include, super elastic NiTi, shape memory NiTi, Ti—Nb, Ni—Ti approx. 55-60 wt. % Ni, Ni—Ti—Hf, Ni—Ti—Pd, Ni—Mn—Ga, Stainless Steel (SST) of SAE grade in the 300 to 400 series e.g., 304, 316, 402, 440, MP35N, and 17-7 precipitation hardened (PH) stainless steel, other spring steel or other high tensile strength material or biocompatible metal material. Examples of polymers include polyimide, PEEK, nylon, polyurethane, polyethylene terephthalate (PET), latex, HDHMWPE and thermoplastic elastomers.

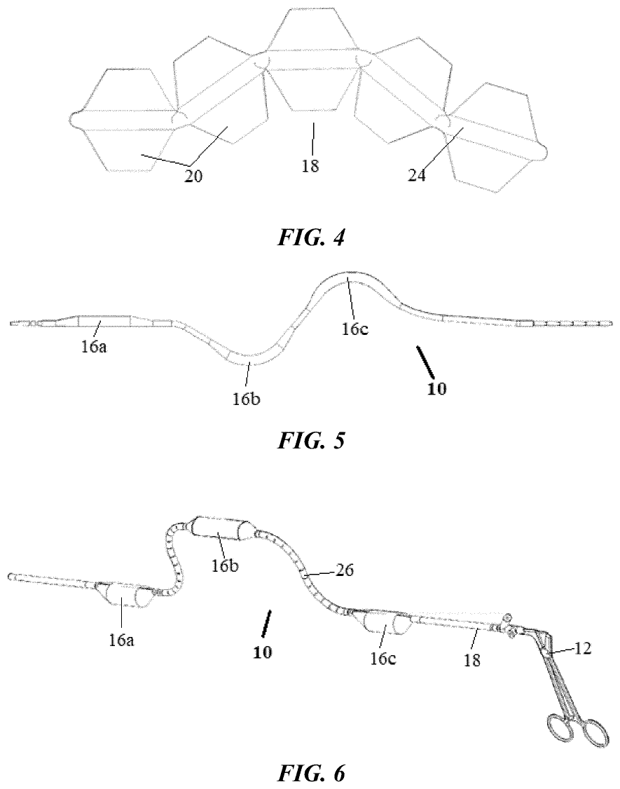

Now referring to FIGS. 2-4, an alternative example of the flexible conduit 18 is shown. In this illustrated example, the conduit 18 may include one or more interconnected, contiguous, and/or unitary geometric components 20 that are movable or pivotable about each other through one or more hinges or pivot segments 22. The hinges or pivot segments 22 may include living hinges that constitute a contiguous portion of the conduit 18 along with the geometric components 20. The individual hinges 22 may alternate in their orientation or angular offset with respect to each preceding and/or subsequent hinge 22 along a length of the conduit 18. For example, each living hinge may be angularly offset between approximately 70 degrees and approximately 110 degrees with respect to the nearest living hinge of the plurality living hinges. In the illustrated example, the angular offset between two successive hinges 22 is approximately 90 degrees.

The geometric components 20 may include substantially cylindrically-shaped bodies with one or more angled faces or portions thereon to provide varying degrees of articulation and range of travel, which is mechanically limited by abutting portions of adjacent geometric components 20.

The resulting combination of the geometric components 20 and the hinges 22 provide a conduit 18 that is flexible in one or more planes, has a selectable degree of resistance to axial compression (e.g., by varying the size, shape, and/or orientation of the hinges 22 and the geometric components 20), and provides a high degree of torque transmission whether the conduit 18 is in a substantially linear configuration (such as that shown in FIG. 2) or in a multi-planar, contoured configuration (such as that shown in FIG. 3). The geometric components 20 also reduce the likelihood of kinking or obstructing an internal lumen or passage 24 extending therethrough, which may be used to transport fluid, wires, or other components therein along a length of the medical device 10.

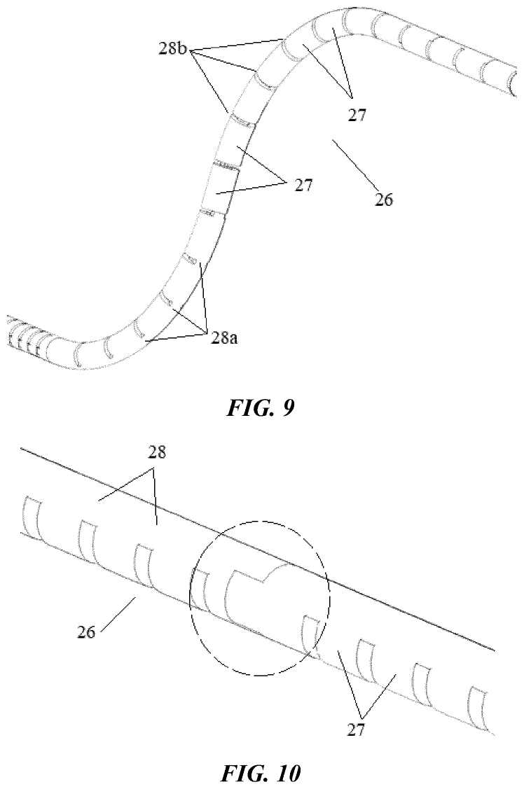

Referring now to FIGS. 5-9 (in which one or more outer layers are removed from the device shown in FIG. 1 for the sake of illustration), the elongate body 14 may include at least one shaping structure 26 coupled to the distal segment of the flexible conduit 18 that is configured to transition from a substantially linear configuration to a predetermined, pre-set, and/or biased curvilinear configuration and/or a predetermined, pre-set, and/or biased configuration where a portion of the shaping structure 26 (and/or the elongate body 14) is laterally displaced from remaining portions of the shaping structure 26 (and/or the elongate body 14) upon the application of an axial compression force to the shaping structure 26 (and/or the elongate body 14). In one example, the shaping structure 26 may include a substantially contiguous support element or spine defining or including a plurality of articulating elements 27 that extend substantially across the entire length of the displaced or shape-modified portion of the elongate body 14. Having a substantially contiguous or unitary structure provides a high degree of torque transmission (e.g., up to a substantially 1:1 proximal-to-distal torque transmission) and thus, improved control of the positioning and orientation of the device 10 within a particular anatomical position. The substantial continuity of the shaping structure 26 may be attained through manufacturing a substantially single, unitary length of material that comprises the entire shaping structure 26, or alternatively, the shaping structure 26 may include several discrete lengths of material that are interlocked or otherwise functionally adhered or assembled to one another to form the substantially contiguous body of the shaping structure. An example of an interlocking joint having matable tabs and protrusions is shown in FIG. 10. In one embodiment, there may be 2, 3, 4, 5, 6, 7, 8, 9, 10 . . . n shaping structures 26 interlocked together.

The shaping structure 26 may include one or more structural and/or material characteristics that allow the device 10 to selectively transition from a substantially linear configuration (such as that shown in FIG. 1) to one or more curvilinear and/or displaced configurations (such as those shown in FIGS. 5-9) in one or more planes upon the application of an axial and/or compressive force. By way of non-limiting example, such curvilinear configurations may include substantially “S”-shaped (such as in FIG. 5), substantially “C”-shaped, and/or substantially “U”-shaped orientations. Alternatively, the shaping structure can assume other configurations in one or more planes such as a corkscrew, a loop or other geometric patterns. The shaping structure may be constructed from at least one of the following, plastics, polymers, silicone, nylon or the like. The shaping structure 26 may include multiple (i.e., a plurality) 2, 3, 4, 5, 6, 7, 8, 9, 10, 11, 12, 13, 14, 15, 20, 30, 40 up to n living hinges 28 that are longitudinally and angularly positioned/offset on the shaping structure 26 to provide the desired shape when compressed or under an axial load.

For example, the shaping structure 26 may include a first plurality of living hinges 28a longitudinally spaced along a proximal portion of the shaping structure 26. The first plurality 28a may provide at least one of a turn or an arc of approximately 90 degrees with respect to a proximal and/or linear segment of the shaping structure 26, elongate body 14, and/or the flexible conduit 18 when an axial compression force is applied. A second plurality of living hinges 28b may be longitudinally spaced along a length of the shaping structure 26 adjacent to and radially offset from the first plurality of living hinges 28a. The radial offset of the second plurality of living hinges 28b provides a varying direction of contour and/or shape compared to the first plurality of living hinges 28a. The range of the radial offset between adjacent hinges may range from about 0 degrees to about 360 degrees between living hinges, e.g., 1, 5, 10, 20, 30, 40, 50, 60, 70, 80, 90, 100, 150, 200, 300, etc. For example, the second plurality of living hinges 28b illustrated in FIGS. 6-9 provide at least one of a turn or an arc of approximately 90 degrees with respect to a proximal and/or linear segment of the shaping structure 26, elongate body 14, and/or the flexible conduit 18, but in an opposite direction compared to the turn or arc of the first plurality of living hinges 28a. In an exemplary embodiment, the radial offset of the pluralities of living hinges described herein may be between approximately 150 degrees and approximately 210 degrees. In the illustrated example, the radial offset is approximately 180 degrees, and the combined span of the first and second pluralities of living hinges thus provide an arcuate, curvilinear, substantially “S”-shaped contour in substantially a single plane.

The shaping structure 26 may further include a third plurality of living hinges 28c that is positioned distally of, and, in one embodiment, substantially radially aligned with, the second plurality of living hinges 28b. The shaping structure 26 may include a fourth plurality of living hinges 28d distally of the third plurality of living hinges 28c, and substantially radially aligned with the first plurality of living hinges 28a. The third and fourth pluralities of living hinges thus provide a curvilinear shape inverted or mirrored with respect to that of the first and second pluralities of living hinges 28a, 28b, as shown.

The shaping structure 26 may include one or more segments that remain in a substantially linear configuration when under an axial load to create the desired geometry or displacement. Such segments may be substantially devoid of living hinges or other bending or contour-inducing features. For example, in the illustrated embodiment, the shaping structure 26 includes a segment 30a positioned between the second and third pluralities of living hinges 28b, 28c, that maintains a substantially linear configuration. The shaping structure 26 may also include substantially linear segments at proximal (30b) and distal (30b) positions along the length of the device 10. In the illustrated example of FIGS. 6-9, the construct of the device 10 provides for lateral displacement of the segment 30a while segment 30a remains substantially parallel to the proximal and distal segments 30b, 30c.

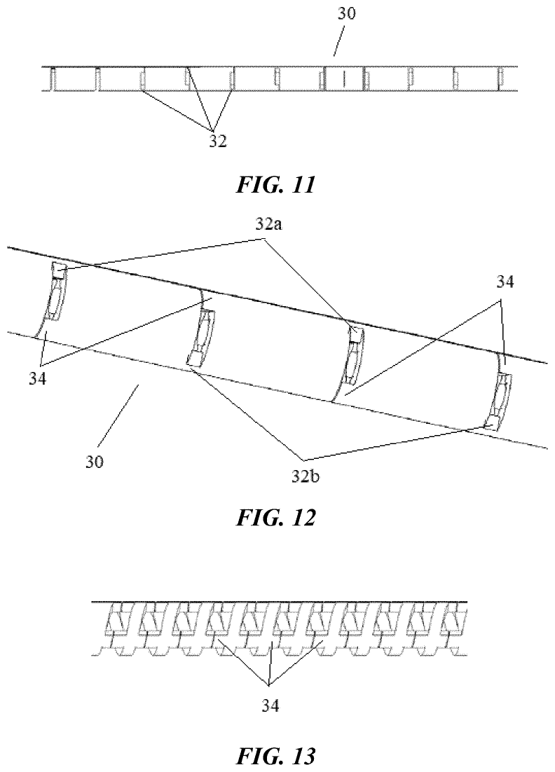

The segments 30a, 30b, and/or 30c (collectively, “30”) may have alternative constructions to provide desired degrees of flexibility in one or more planes, but resist bending or contouring when under an axial load. For example, as shown in FIGS. 11-12, the segment 30 may include a plurality of living hinges 32 longitudinally spaced along a length thereof. The plurality of hinges 32 may include subsets of hinges 32a, 32b that alternate with one another and have varying angular offsets along the length of the segment 30. For example, in the illustrated example, the hinges 32a are angularly offset from the hinges 32b by approximately 180 degrees, which restricts bending of the segment 30 to a single plane. Other angular offsets may be implemented to provide desired degrees of flexibility and bending in one or more planes.

In addition to the hinges 32, the segment 30 may include a plurality of stopping elements 34 that restrict a motion range or degree of bending for a particular hinge 32. For example, each stopping element may include a protrusion or other mechanical feature that can abut an opposing surface or component to resist further movement. Each stopping element 34 may be longitudinally aligned with an individual hinge 22, but radially offset from each hinge 22 by approximately 180 degrees such that the stopping element 34 does not interfere with the hinge bending or flexing in a first direction (e.g., in a direction that moves the stopping element away from the adjacent, abutting surface), but restricts movement or bending of the hinge in a second direction substantially opposite to the first direction (e.g., the direction in which the stopping element 34 is moved to abut the opposing surface). The illustrated combination of the radial offsets and stopping element 34 provides flexibility in a single plane, but resists bending or contouring when under an axial load.

Now referring to FIG. 13, another example of the segment 30 may include a plurality of stopping elements 34 arranged about a spiral configuration that provides flexibility in multiple planes, but resists bending or contouring when under an axial load.

In addition to restricting flexion or bending of certain segments of the device 10, the stopping elements 34 may add to a torsional rigidity to one or more segments of the device 10. For example, one or more stopping elements may include a plurality of teeth, a crown, ridges, tabs, and/or slots (not shown) that engage complementary features or structures on an opposing surface or component of the segment, such that the complementary features interlock or engage each other when an axial force is applied to the segment. The releasably interlocking nature of the respective, complementary features then resists rotational movement between the interlocked components, and thus provides a high degree of torsional rigidity and torque transmission along the length of the segment.

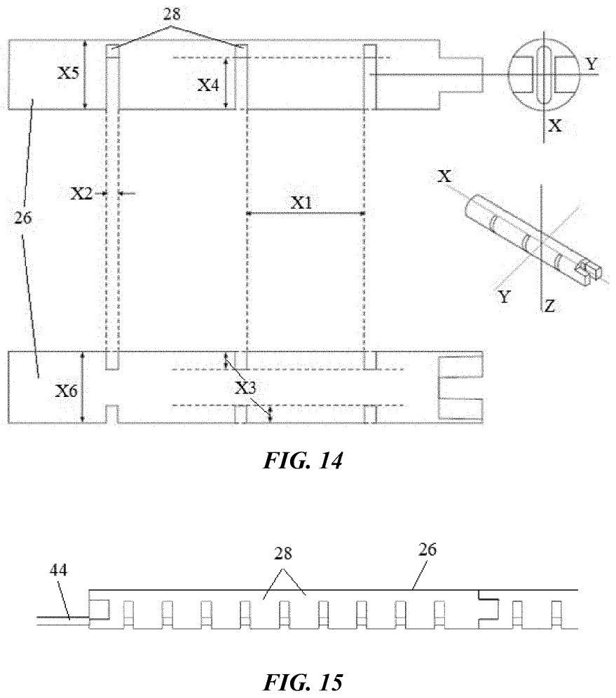

The living hinges in the example of the device 10 shown in FIGS. 6-9 include a substantially square or rectangular-shaped portion of material, interlocking individual adjacent articulating elements, 27, of the shaping structure 20. Various features of these hinges and the surrounding structures may be modified to attain desired shapes and degrees of flexibility of the shaping structure 26. For example, now referring to FIG. 14, such variable characteristics may include: the longitudinal distance X1 between consecutive hinges; the width X2 of a space or carve out (e.g., gap) between articulating segments of the shaping structure 26; the depth X3 of the cross-sectional portion removed surrounding the hinge; the height X4 of the gap or space underneath the hinge and between articulating segments; the overall height X5 of the shaping structure 26; and/or the overall width X6 of the shaping structure 26.

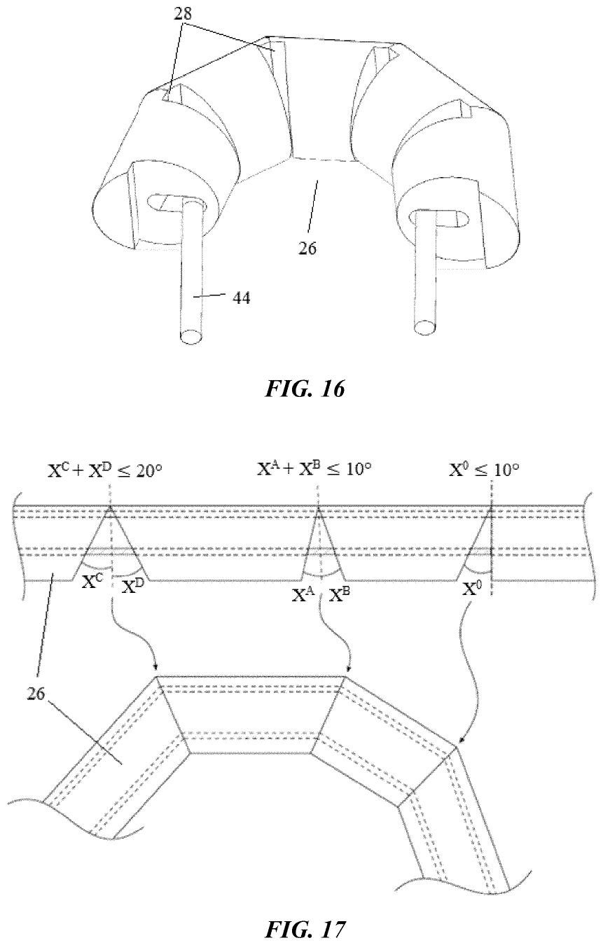

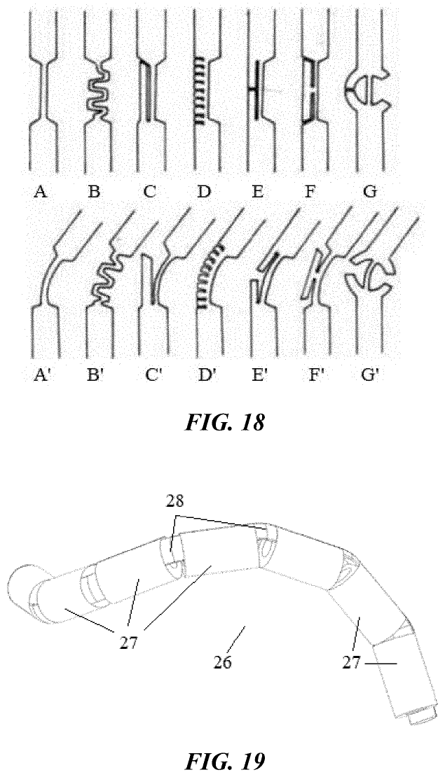

The shape of the hinges and/or gap or space between adjacent articulating segments may also include and/or vary amongst rectangular (such as in FIG. 15), trapezoidal (such as in FIG. 16), triangular (such as in FIG. 17), rhomboidal, circular or arcuate, or the like. Angled features or characteristics of the hinges may also be varied to provide varying degrees and directions of bending and/or flexibility. For example, as shown in FIG. 17, varying the angle of the walls of two adjacent articulating elements 27 varies the resulting distance or pivoting range that the articulating element travels under axial compression, and thus can be varied to attain the desired geometric configuration. The angle of the walls can vary from about 0 degrees to about 70 degrees, wherein the angle is measured by a hypothetical plane bisecting the tube at a 90-degree angle relative to the longitudinal axis of the tube.

Additional alternative examples of living hinge constructions that may be implemented to achieve the configurations and features disclosed herein are illustrated in FIGS. 18A-G in the unstressed configuration, and FIGS. 18A′-G′ in the contoured or bent configuration under a load.

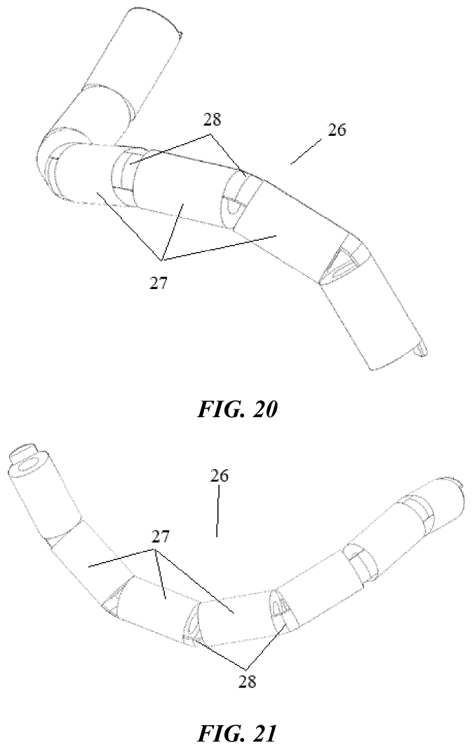

Now referring to FIGS. 19-21, an example of a multi-planar configuration of the shaping structure 26 is shown from varying viewpoints. As shown, the angular offset of the hinges 28 varies incrementally from one hinge to the next to provide a multi-planar configuration when the shaping structure 26 is placed under an axial load, thereby causing the articulating elements 27 to pivot about the hinges 28 and into contact with each other to complete the geometric transformation. The illustrated example demonstrates the multi-planar capability of the present disclosure, which can provide a myriad of different shapes, contours, bends, and turns for the device 10.

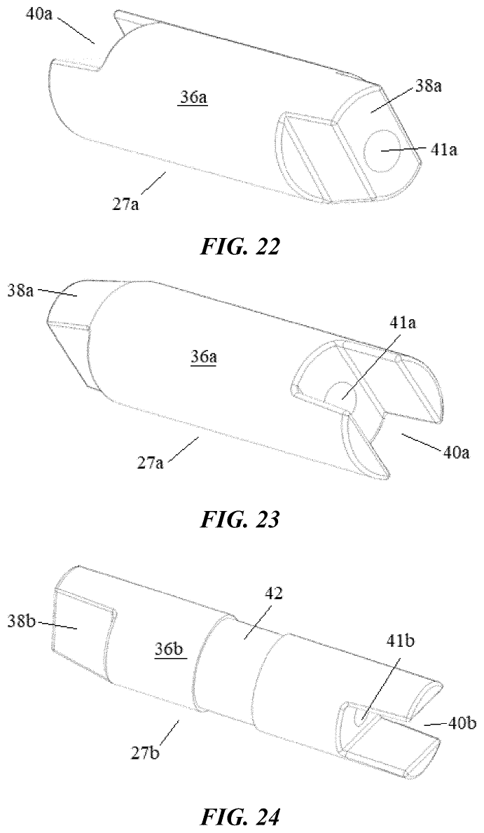

Now referring to FIGS. 22-26, an example of a shaping structure 26 is shown that is constructed from multiple, discrete articulating elements 27 that create a plurality of pivoting or hinging joints to form varying geometric patterns, shapes, contours, or the like in one or more planes. As shown in FIGS. 22-23, the shaping structure may include a first variant of an articulating element 27a that generally defines or includes a body 36a having a protruding portion 38a at one end of the body 36a and a slot or recessed cavity 40a opposite of the protruding portion 38a. The body 36a may define a substantially cylindrical shape, and may have one or more lumens or passages 40a extending therethrough. The protruding portion 38a may have one or more tapered sides or surfaces that are complimentary to interlock with or otherwise positioned within the recessed cavity 40a when coupling multiple, articulating elements. The complimentary features of the protruding portion 38a and the recessed cavity 40a are axially aligned and substantially parallel to the articulating element 27a.

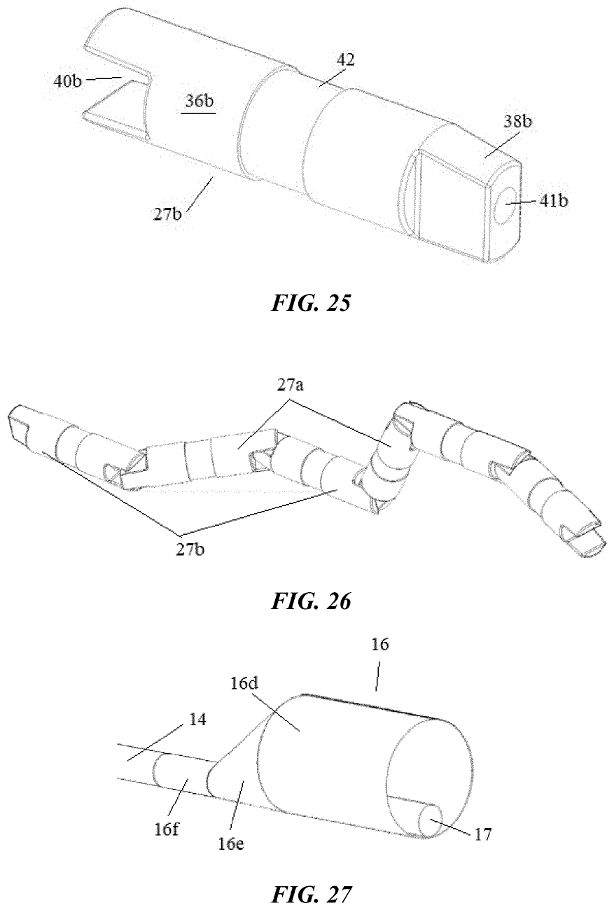

As shown in FIGS. 24-25, the shaping structure may include a second variant of an articulating element 27b that generally defines or includes a body 36b having a protruding portion 38b at one end of the body 36b and a slot or recessed cavity 40b opposite of the protruding portion 38b. The body 36b may define a substantially cylindrical shape and may have one or more lumens or passages 40b extending therethrough. The bodies 36a and/or 36b may also include or define a depressed surface area or reduced outer dimension region 42 for receiving a marker band, c-clamp, or other mechanical components to facilitate operation or assembly of the device 10. The protruding portion 38b may have one or more tapered sides or surfaces that are complimentary to interlock with or otherwise positioned within the recessed cavity 40b when coupling multiple articulating elements. In the embodiment shown, the complimentary features of the protruding portion 38b and the recessed cavity 40b are substantially perpendicular to each other in the articulating element 27b.

As shown in FIG. 26, the articulating elements 27a, 27b may be interconnected to provide multi-planar configurations due to the parallel and perpendicular orientations of the respective protruding portions 38 and the recessed cavities 40 along the length of the formed shaping structure 26. Numerous shapes and configurations can be attained through the interlocking use of varying articulating elements 27a, 27b. Additional variations in the respective angular positioning or orientation for the protruding portions 38a, 38b and the recessed cavities 40a, 40b may be introduced to achieve a desired configuration (e.g., in addition to and or alternatively to the illustrated aligned or perpendicular orientations, one or more of the articulating elements 27 may have an angular orientation of its protruding portion and recessed cavity set at any value between 0 and 90 degrees).

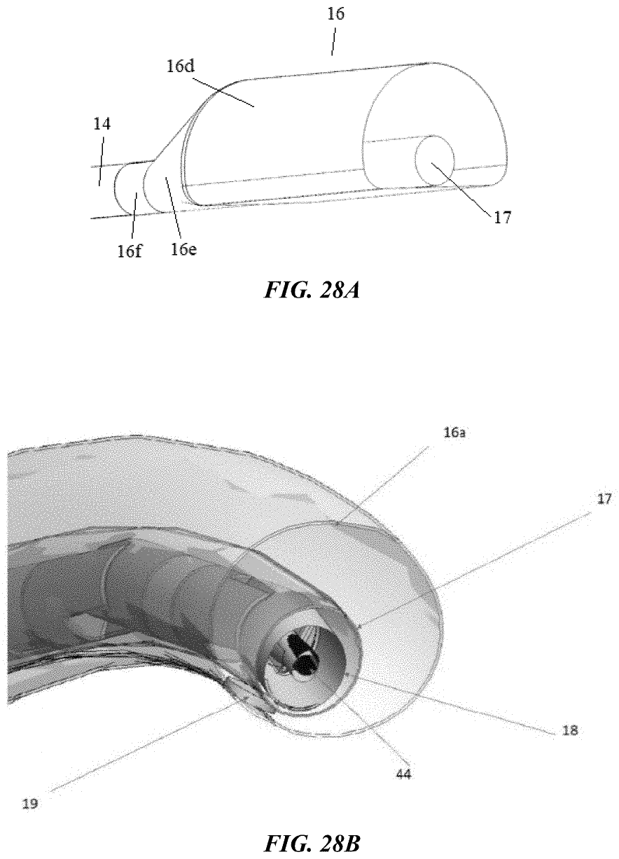

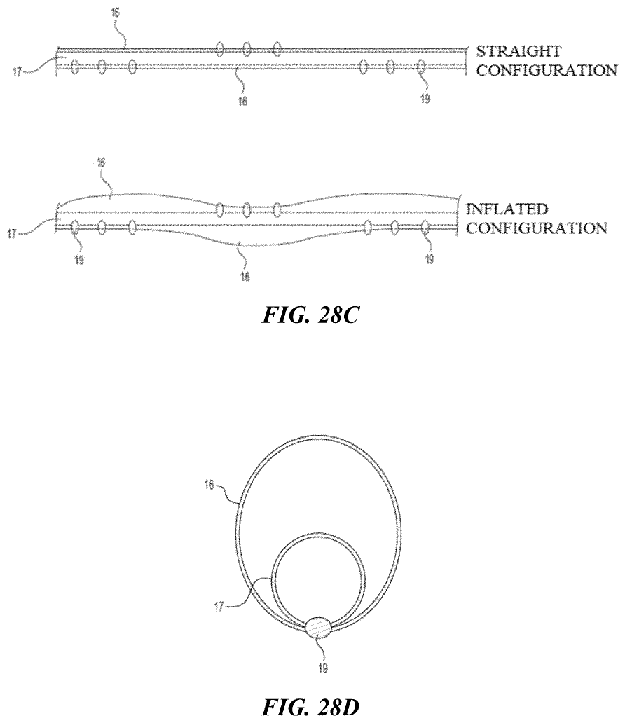

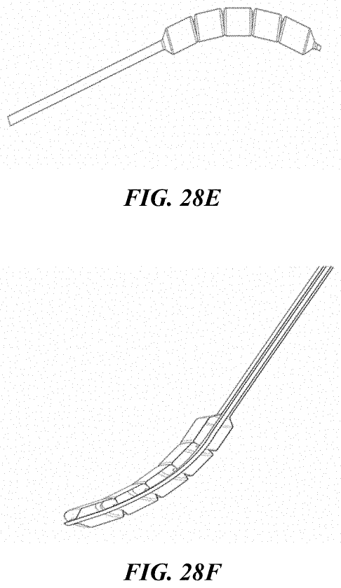

As described above, the device 10 may include one or more balloons 16 (16d—balloon body, 16e—balloon shoulder, 16f—balloon leg) positioned along a length of the elongate body 14 and/or the shaping structure 26. A balloon assembly inner body 17 is positioned in co-axial arrangement over the shaping structure 26. The balloons 16 may be anchored or otherwise secured to the balloon assembly inner body 17 by one or more spot welds 19, heat fusions, clamping rings, adhesive, or other means to secure the connection between these components, and to reduce or eliminate any axial movement between the balloons 16 and the shaping structure 26 during use. If there are two or more anchors, e.g., spot welds 19, the spot welds 19 are positioned asymmetrically at one point on the balloon. Because the balloon 16 is anchored either at one point, i.e., one spot weld 19, or asymmetrically, i.e., a plurality of spot welds 19, the balloon 16 expands or inflates in an asymmetric manner, FIGS. 28A and B in one direction. For example, the cross-sectional lumen of the balloon can assume a semi-circular or partially-circular, cross-sectional area such as an elliptical or oval cross-section, FIGS. 28A and B. The balloon 16 may also have a substantially flattened surface segment when inflated or expanded, an example of which is shown in FIGS. 28A and 28B. This asymmetric expansion or inflation provides a means for the balloon 16 to support, cushion, contact, and/or exert a force on a tissue region. In one embodiment, the balloons may be formed from a segmented balloon structure. This segmented structure allows the balloon to conform to the structure of the shaping element. FIG. 28E is an illustration of a segmented balloon. FIG. 28F is a cross-sectional illustration of FIG. 28E.

The balloons may be constructed from one or more elastically expandable, i.e., compliant, and/or non-plastically deformable materials, i.e., noncompliant, such as nylon, polyurethane, or the like, and/or may be constructed or include one or more radiopaque or radiation shielding materials.

One or more of the balloons 16 may be asymmetrically expandable about only a portion of the circumference of, and/or have a non-concentric mounting on, the elongate body 14 and/or shaping structure 20, an example of which is shown in FIG. 27. These non-concentric and semi-circular balloon configurations increase the distance that the inflated surface of the balloon travels away from the longitudinal axis of the elongate body 14 and/or the shaping structure 26 in a targeted direction, rather than expanding equally in all directions about a circumference of the elongate body 14 and/or the shaping structure 26 if concentrically-oriented balloons were employed. These features thus, in turn, increase the ability of the device 10 to contact and displace tissue away from a longitudinal axis of the device while reducing the risk of stretching or deforming an overall circumference of the adjacent tissue.

One or more of the balloons 16 may be angularly offset compared to one or more of the remaining balloons 16 of the device between approximately 100 to approximately 250 degrees or from about 150 degrees to about 210 degrees. In the illustrated device of FIGS. 1 and 5-7, the balloon 16b is angularly offset from the balloons 16a, 16c by approximately 180 degrees. The range of angular offset between two balloons may vary for a particular procedure or use. Alternatively, one of the balloons 16 may wind or spirally wrap around the elongate body 14 and/or the shaping structure 26 such that a single balloon provides varying surface segments that are angularly offset from other surface segments of the same balloon. FIG. 28C.

The balloon(s) 16 may be mounted or adhered to the elongate body 14 and/or the shaping structure 26 in numerous ways to provide a reduced profile for packaging, insertion, delivery, and/or positioning of the device in a particular medical procedure. For example, the balloon(s) 16 may be folded or pleated to reduce an overall circumferential profile. The balloon(s) 16 may subsequently be controllably inflated and/or deflated through the introduction of an inflation medium (e.g., air, nitrogen, radiopaque contrast medium, saline, etc.) through one or more ports at a proximal portion of the device 10, as described below. The balloon(s) 16 may be individually inflatable independently through an assigned inflation lumen or inflated substantially simultaneously through a single, all-balloon-encompassing inflation port. Such inflation characteristics may be facilitated through one or more fluid passages, valves, controllers, sensors, or the like located on or about portions of the device, and/or in communication with one or more portions or components of the device 10. The balloon(s) 16 and/or the device 10 may also include one or more sensors or features to monitor, assess, and/or alert an operator regarding performance or situational characteristics of the balloon(s) 16 and/or device 10, including for example, contact with tissue, inflation pressure, fluid flow, temperature, impedance or other electrical activity, or the like.

In addition, and/or alternatively to the balloon(s) 16, the device 10 may include one or more non-inflatable cushioned elements positioned to contact, displace, and/or otherwise disperse force across a targeted tissue area. Such cushioned elements may be constructed from or otherwise contain pliable materials, polymers, or the like, such as silicone, rubber, sponge-like materials, gels, hydrogels, or the like.

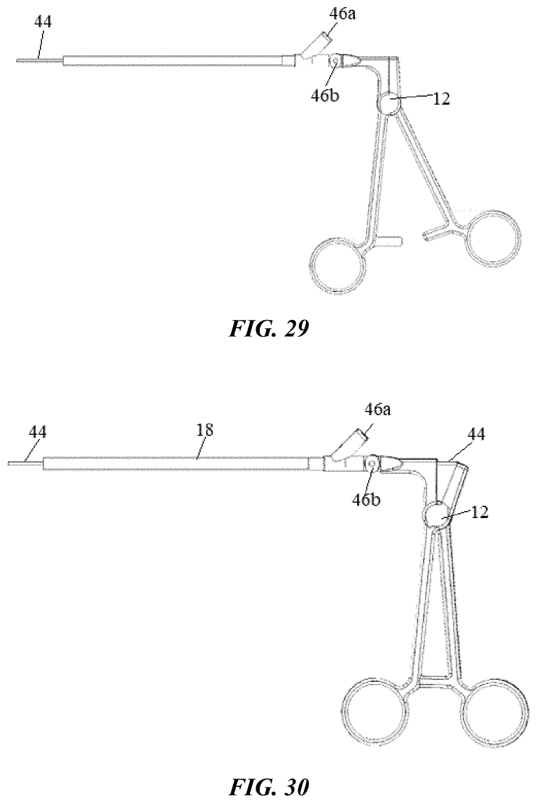

The handle 12 at the proximal portion of the device 10 allows for selective adjustment of the geometric configuration of the device. Now referring to FIGS. 29-30, the handle 12 may generally include one or more actuation or control features that allow a user to control, deflect, steer, or otherwise manipulate a distal portion of the medical device 10 from the proximal portion of the medical device. In the illustrated example, the handle 12 includes a forceps-like interface that can be selectively opened, closed, and/or maintained (e.g., through a ratchet-like mechanism) to actuate a pull wire 44. It will be understood that the pull wire 44 may be coupled to the device 10 in any manner suitable to create an axial force or load on the elongate body 14 and/or the shaping structure 26. Alternative operable examples for the handle 12 may include a knob, wheel, lever, threaded actuator, plunger, or the like that is movably coupled to a proximal portion of the elongate body 14 and/or the handle 12, and which may further be coupled to the pull wire 44 such that manipulating the knob, wheel, lever, or the like exerts a force upon the pull wire 44.

The handle 12 may include an “open” position that exerts minimal or marginal force upon the pull wire 44 (and thus the elongate body 14 and/or the shaping structure 26), as shown in FIG. 29, and could correspond to the substantially linear configuration of the device 10 shown in in FIG. 1. An example of a “closed” position in which axial forces are exerted on the pull wire 44 (and thus the elongate body 14 and/or the shaping structure 26) by the handle 14 is illustrated in FIG. 30, which may correspond to the geometrically-transitioned configuration of the device 10, such as that shown in any of FIGS. 3-9, 15-17, 19-21, and/or 26.

In addition and/or alternatively to the ratchet-like mechanism shown and described above, the handle 12 may further include one or more features or mechanisms to maintain a particular force and/or displacement of the pull wire 44, such as a threaded collar or other locking mechanism, a gear assembly, a set screw, and/or clamping or other tensioning elements. The handle 12 may include a visual reference indicator that indicates the direction of deflection or displacement of segments of the device, and/or indicators of the axial load or force being exerted upon the device.

The handle 12 and/or a proximal portion of the device 10 may include one or more ports 46a, 46b for the introduction of one or more materials, compounds, mediums, or otherwise into internal portions of the device 10. For example, the port 46a may be in fluid communication with an interior of one or more of the balloons 16 for the introduction or exhaustion of an inflation medium or fluid, while port 46b may be implemented for the introduction of a contrast agent (media) or flushing solution to facilitate a particular procedure being performed. Port 46b may be in fluid communication with another exit port or vent which is positioned along elongate body 14 which allows for introduction of contrast media or flushing solutions into the lumen, i.e., body cavity, where the device is situated, for example, the esophagus.

The pull wire 44 may extend along substantially an entire length of the elongate body 14 and have a distal end anchored to one or more components towards the distal region of the device 10. In one or more alternative configurations, the device 10 may include multiple pull wires that are independently controllable and/or anchored at different points along the length of the device to provide for multi-stage operation to achieve differing shapes and/or to manipulate the configuration of discrete portions of the device.

The pull 44 wire may be constructed from one or more polymers, plastics, metals, and/or composites or combinations thereof. The pull 44 wire may be composed of a braided cable, where the cable is composed of various polymers and/or metals. The pull wire 44 may have material properties providing for a predetermined or preset tension limit or threshold, such that the pull wire 44 breaks or deforms prior to reaching or exceeding a tension or force amount that could damage other components of the device (including, for example, the shaping structure 20 or portions thereof) and/or exert traumatic forces onto surrounding tissue structures. The pull wire 44 may thus provide a degree of safety during use to mitigate any excessive forces and resulting potential to damage surrounding tissue areas.

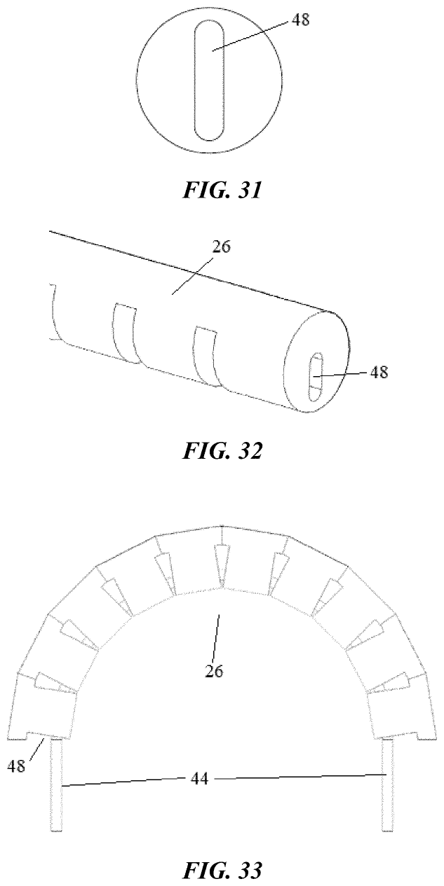

The shaping structure 26, flexible conduit 18, and/or other portions of the elongate body 14 may include one or more lumens 48 therethrough for operable components such as pull wires, e.g., cables, fluid conduits, guide wires, electrical wires, or the like. Now referring to FIGS. 31-35, examples of cross-sectional geometries for such components are shown. In the illustrated examples of FIGS. 31-32, each lumen 48 includes an elongated or oblong shape extending across a substantial width or diameter of the respective component (e.g., the shaping structure 26, conduit 18, or elongate body 14). In the embodiment shown, the elongated span of the lumen 48 provides a mechanical advantage by increasing the cross-sectional distance between a pull wire and a hinge or pivot point that the pull wire is acting upon when transitioning the device 10 from a substantially linear configuration to a curvilinear/contoured configuration, as described herein. In the example illustrated in FIG. 32, the lumen 48 is offset from the center of the cross-section, away from the location of the hinges 28 to allow the pull wire to achieve an even greater mechanical advantage during use, as illustrated in FIG. 33.

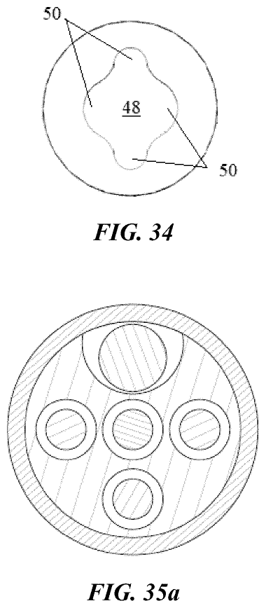

Now referring to FIG. 34, the lumen 48 may have a contoured cross-sectional profile defining multiple recesses or pockets 50 (the illustrated example includes 4 such pockets positioned approximately at 0 degree (i.e., 12 o'clock), 90 degree (i.e., 3 o'clock), 180 degree (i.e., 6 o'clock), and 270 degree (i.e., 9 o'clock) positions) that reduce the distance between the pull wire 44 and circumferential edge or surface that the pull wire 44 is moved towards when the device 10 is under an axial load such as when the device 10 is in a no-linear configuration. The multiple pockets 50 allow the pull wire to transition to different pockets along the length of the pull wire, elongate body 14, and/or the shaping structure 26 in conjunction with varying hinges that are radially offset along the longitudinal length of the device 10. For example, in one longitudinal segment of the elongate body 14, and/or the shaping structure 26, the pull wire 44 under axial load may move into the pocket 50 at the 0 degree location, while in a more distal longitudinal segment of the elongate body 14, and/or the shaping structure 26, the pull wire 44 under axial load may move into the pocket 50 at the 90 degree location due to the different curvature in that distal longitudinal segment when the device 10 is under an axial load.

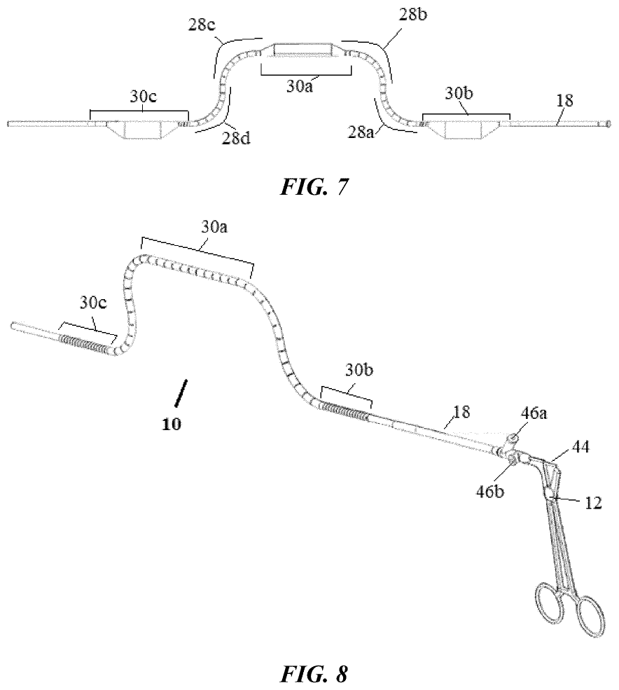

The cross-sectional position of the examples of the lumens 48 disclosed herein may change along the length of the elongate body 14 and/or the shaping structure 26 such that the mechanical advantage of the offset of the lumen 48 and pull wire 44 from the hinges or other articulating point of the device 10 remains substantially constant (or within a particular distance range) throughout the length of the device 10 for varying hinge orientations having varying angular offsets as described herein. For example, in the device illustrated in FIG. 7, the segment having the hinges 28a may include the lumen 44 positioned towards an outside surface of the device 10 away from the living hinges 28a, while the segment of the device having the living hinges 28b has the lumen 44 transitioned towards an inner surface of the device opposite the hinges 28b.

The off-center lumens and resulting position of the pull wire 44 not only provides mechanical advantages to exert a bending force on the respective living hinges 28 or articulating elements 27 along the length of the device 10, but also, provides increased torsional rigidity when the device 10 is in a compressed, geometrically-transitioned configuration. When under a torsional load, the living hinges 28 and/or articulating elements 27 would torque and twist around their connection point—which in several of the illustrated examples would be the living hinge 28 running along an exterior surface of the device 10, thereby resulting in a twisting and rotational displacement occurring between each articulating element 27. However, the off-centered lumens and pull wire 44 add rigidity and alignment to the surface opposite the living hinges in each articulating segment, thereby balancing torsional forces more towards the centerline or longitudinal axis of the device 10. As a result, the articulating segments and the shaping structure turns to transmit torque as a substantial whole or cylindrical entity, rather than having twisting and rotational displacement between each articulating element.

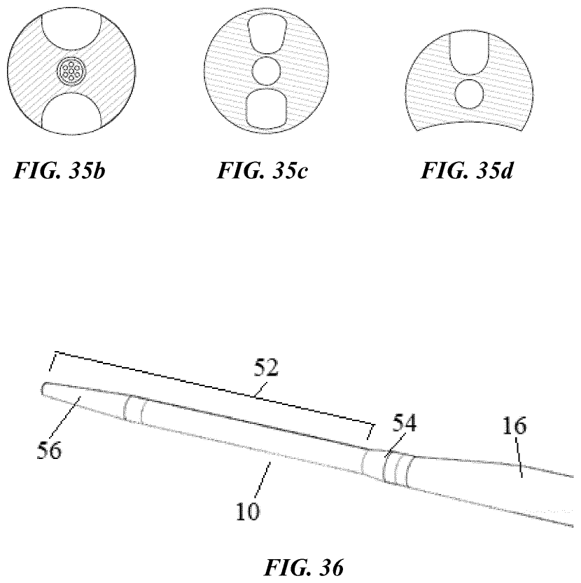

FIGS. 35A-D illustrate additional examples of varying lumen configurations for one or more components traversing lengths or segments of the medical device 10.

The device 10 may include one or more segments that are not tensioned or placed under axial load when the handle 12 and/or pull wire 44 are tensioned. For example, now referring to FIG. 36, the device may include a distal end section 52 that is distal of the balloon 36 and distal of the point, segment, or region 54 where the pull wire may be coupled to the elongate body 14 and/or the shaping structure 26. As the distal end section 52 is outside of the operative axial loading of the pull wire 54, the section 52 remains flexible and/or pliable irrespective of the loads and/or geometric configurations of the other segments of the device 10. During use of the device 10, the physiological pliability or flexibility of the distal section 52 avoids exerting pressure or forcefully contacting tissue that is distal or otherwise remote from the particular tissue being targeted for displacement and/or treatment. The distal end section 52 may include an atraumatic tip 56 that is tapered, conical, or otherwise narrower than other sections of the device 10 and/or the distal end section to aid in navigating and positioning the device 10 in a desired location and/or orientation within an anatomical vessel, cavity, or the like. The tip 56 may be constructed form one or more pliable or relatively soft materials, such as silicone, rubber, or other polymers, and/or the tip may include radiopaque or radiolabeled material therein to aid in imaging and use.

The device 10 may include one or more exterior layers, sheaths, or covers that seal, protect, and/or facilitate use of the device 10 and/or form a portion of the elongate body 14. Such components may include one or more polymer layers fused, adhered, or otherwise permanently affixed to one or more of the components of the device 10, such as the shaping structure 26, the handle 12, one or more of the balloons 16, and/or the distal end section 52. In addition, and/or alternatively to one or more permanently affixed layers, a removable sheath or cover may be used to encapsulate or envelope one or more portions of the device 10 for a procedure, with the sheath or cover being disposed of after the procedure. The device 10 may then be re-used with a new, sterile sheath or cover for a subsequent procedure.

The device 10 may include and/or otherwise be operable with various monitoring, detecting, and/or treatment modalities and respective components and accessories. For example, a temperature sensitive monitoring element may be positioned on the device 10. A radio frequency or current sensitive monitoring element may also be positioned on the device 10. Additionally, a luminal mapping element may also be positioned on or about the assembly of the device 10. This mapping system may have a mapping element which can be manipulated along the longitudinal axis of the device (for example through a lumen running primarily from proximal to distal within or about the segmented device) to enable mapping of the luminal tract without having to move or reposition the device.

The device 10 can incorporate an esophageal temperature probe. Pacing or heart stimulating electrodes can also be incorporated in addition to sensors (e.g., temperature sensors). The pacing electrodes may be placed on the probe and configured to be in contact with the wall of the esophagus. The pacing electrodes may either be bi-polar or mono-polar electrodes. For example, the pacing electrodes may be individually coupled to a radiofrequency (“RF”) generator with selection circuitry to enable individual or multiple electrodes to be selected for use. The electrodes may also be able configured and coupled to electrophysiology monitoring equipment to sense heart electrical activity. The esophageal probe may include or be configured to electrically couple to an interface circuit that is configured to shut off the RF generator if the measured patient temperature does not meet a predetermined threshold. For example, if the patient's temperature exceeds a high-temperature threshold or falls below a low-temperature threshold (which may be useful when the procedure includes cryogenic treatments).

The device 10 may incorporate radiopaque markers for aiding radiographic visualization of the positioning of the device in the esophageal lumen. The markers can include a radiopaque material, such as metallic platinum, platinum-iridium, Ta, gold, etc., in the form of wire coil or band, vapor deposition deposits, as well as radiopaque powders or fillers, e.g., barium sulfate, bismuth trioxide, bismuth sub carbonate, etc., embedded or encapsulated in a polymer matrix. Alternatively, the markers can be made from radiopaque polymers, such as radiopaque polyurethane. For example, the markers can be in the form of bands or partial bands to encircle an outer sheath, shaping element 26, along the elongated patent or layer of the distal section 52.

The radiopaque markers may be configured as bands. Alternatively, the markers can be configured as surface patches. The radiopaque markers should have sufficient size and suitable configuration/construction (e.g., the type of radiopaque material, load amount of radiopaque material, etc.) such that they can be visualized with the proper radiographic aid.

The shaping structure 26 and/or other components of the device 10 may be manufactured from 3D printing processes to provide the features shown and described herein. Rapid prototyping, additive manufacturing, or 3D printing processes utilize a three-dimensional (3D) CAD file to produce a 3D object at significantly lower expenses compared to traditional manufacturing methods. Methodologies such as selective-laser-sintering (“SLS”), stereolithography (“SLA”), inkjet printing, and extrusion-based 3D printing or FFF (fused filament fabrication) may be implemented. Several types of low temperature thermoplastic polymers, such as ABS (acrylonitrile butadiene styrene) and PLA (polylactic acid) may be used in addition and/or alternatively to higher-end engineering polymers, such as nylons, polyetheretherketone (PEEK), polyetherketoneketone (PEKK), polyphenylsulphone (PPSU), polycarbonate (PC), and polyetherimide (PEI). One or more fiber fillers, such as carbon, or glass fibers, may be been added to a polymer base material to enhance the mechanical properties of the shaping structure 26 and/or other components being manufactured.

In an exemplary method of use of the device 10, the device 10 may be in a substantially linear configuration where the pull wire 44 and the respective components of the device operably coupled to the pull wire 44 are not under any significant axial load or pressure. The device 10 may be steered or navigated towards a tissue region of interest for displacement and/or treatment, whereby the flexible characteristics of the device as described herein aid in navigating tortuous anatomical paths to reach the targeted tissue area. The approach and positioning of the device 10 may be intravascular, intraluminal, transdermal, percutaneous, or otherwise, and may be assisted or facilitated by one or more imaging modalities. Once the desired positioning has been achieved, the device 10 may be actuated to transition from the substantially linear and/or flexible configuration to the altered geometric configuration under axial load. The transition of the medical device 10 may be achieved, for example, by actuating the handle 12 to exert a force on the pull wire 44, which in turn axially loads the shaping structure 26 to transition into one or more arcuate, contoured, and/or bent configurations. The one or more balloons 16 may be inflated before, during, and/or after the geometric transformation of the shaping structure to contact the targeted tissue area. The geometric transformation of the shaping structure and/or inflation of the balloons can thus exert targeted force onto the targeted tissue area to displace the tissue for subsequent treatment, analysis, or the like.

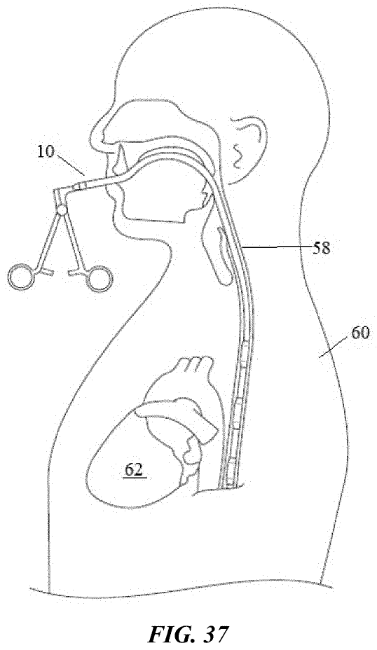

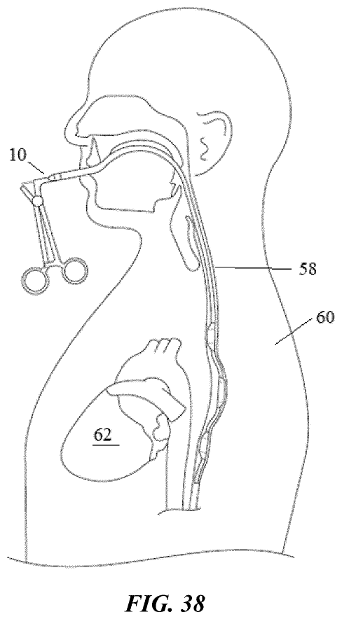

In a particular exemplary use, the device 10 may be used to displace portions of the esophagus away from the heart during the application of thermal or energetic treatments to the heart, such as that associated with arrhythmogenic ablation therapies. Now referring to FIGS. 37-38, the device 10 may be introduced into the esophagus 58 of a patient 60 (either orally or nasally, for example). When introduced and routed into the esophagus, the device may be in the substantially linear and/or flexible configuration, as shown in FIG. 37. The device may be navigated and positioned such that the portion or segments of the device 10 that deflect or transition into a secondary geometric configuration are in a segment of the esophagus adjacent to the heart 62. For example, in FIG. 37, the balloons 16a, 16b, 16c and the middle segment 30a are substantially adjacent to the heart 62. Once in position, the handle 12 may be actuated to tension the pull wire 44 and cause the device to transition to the altered geometric configuration, as shown in FIG. 38. Continuing to refer to the FIG. 38, the altered geometry of the device displaces the affected segment of the esophagus posteriorly away from the heart. The balloons 16 of the device 10 contact the esophagus and provide an increased surface area to disperse the exerted force of the device 10 to reduce or minimize bruising of the esophageal tissue. Having displaced the esophageal segment away from the heart, the thermal and/or energetic treatments of the heart can proceed with a reduced risk of damaging the esophageal tissue.

Another exemplary use of the device 10 may include displacing the esophagus anteriorly into contact with the heart to displace the heart anteriorly and/or laterally away from radiation or other potentially harmful treatments focusing on tumors in the breast. There are an estimated 232,000 new cases of invasive breast cancer and 62,500 cases of breast carcinoma in situ diagnosed each year. Beck et al. Treatment techniques to reduce cardiac irradiation for breast cancer patients treated with breast-conserving surgery and radiation therapy: a review. Frontiers in Oncology, 4(327):2 (2014). Most of these women will receive breast-conserving surgery, followed by radiation. A potentially serious complication of radiation therapy is cardiac toxicity, e.g., radiation delivered to the target tumor bed and/or regional lymph nodes can also intersect the heart. Potential complications arising from this incidental cardiac irradiation can include ischemic heart disease, heart failure, valvular disease, or even death from heart disease. An exemplary method for reducing radiation dosage to the heart involves displacing the heart using the device described herein. For example, the device 10 may be introduced into the esophagus and positioned adjacent to the heart, as described above. The device 10 would then be actuated to transition to the alternative geometric configuration which may direct the device to displace the esophagus anteriorly (rather than posteriorly, as described above), with the device and the esophagus then being moved to contact and displace the cardiac tissue anteriorly and/or laterally out of the damaging field of radiation or therapy.

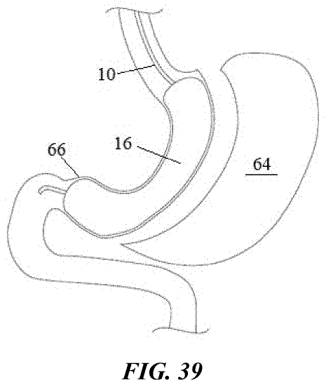

Because of the ability to introduce both curved and comparatively linear sections at any point along the shaping structure 26 of the device 10, another exemplary use of the device 10 may include supporting and/or conforming tissue for gastric tubulization in the stomach during surgical resection. For example, as shown in FIG. 39, the device 10 may be introduced into a segment of the stomach, and a balloon 16 may be inflated along the length of the device 10 to form a shape substantially dictated by a geometrical configuration of the device 10 under axial load. A portion 64 of the stomach may be resected as part of the gastric procedure, while the tissue 66 supported by and/or conformed to the device 10 may be sealed to complete the procedure. The balloon 16 may subsequently be deflated and the device removed.

In another exemplary use, the device 10 may be used to deflect or displace targeted tissue portions during or in anticipation of prostate radiation therapy. For example, the device 10 may be inserted into the urethra to displace one or more tissue segments from a radiation field.

It will be appreciated by persons skilled in the art that the present disclosure is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the examples disclosed herein are not necessarily exclusive of each other and may be included in a variety of different combinations or configurations without departing from the scope and spirit of the disclosure. A variety of modifications and variations are possible in light of the above teachings without departing from the scope and spirit of the disclosure, which is limited only by the following claims.

What is claimed is:

1. A medical device, comprising:
a handle;
a flexible conduit having a proximal segment and a distal segment, wherein the proximal segment is coupled to the handle; and
a substantially contiguous shaping structure coupled to the distal segment of the flexible conduit, wherein the shaping structure is configured to transition from (i) a substantially linear configuration to (ii) a configuration where a portion of the shaping structure is laterally displaced from remaining portions of the contiguous shaping structure upon the application of an axial compression force to the shaping structure, wherein the shaping structure is a unitary spine defining a plurality of radially offset living hinges, and wherein the shaping structure includes:
a first plurality of living hinges;
a second plurality of living hinges radially offset from the first plurality of living hinges between approximately 150 degrees and approximately 210 degrees;
a third plurality of living hinges substantially radially aligned with the second plurality of living hinges; and
a fourth plurality of living hinges substantially radially aligned with the first plurality of living hinges.

2. The medical device of claim 1, wherein the portion of the contiguous shaping structure is laterally displaced substantially within a single plane.

3. The medical device of claim 1, wherein the portion of the contiguous shaping structure is laterally displaced substantially in at least two planes.

4. The medical device of claim 1, wherein the shaping structure includes a segment between the second plurality of living hinges and the third plurality of living hinges that substantially resists bending from the application of the axial compression force.

5. The medical device of claim 4, wherein the segment includes:
a plurality of living hinges extending along a longitudinal length of the segment, wherein each living hinge of the plurality is angularly offset by approximately 180 degrees with respect to a consecutive living hinge of the plurality, and
a plurality of stopping elements, wherein each stopping element is radially offset by each living hinge of the plurality by approximately 180 degrees to restrict a motion range of the respective living hinge.

6. The medical device of claim 1, wherein the first plurality of living hinges provides at least one of a turn and an arc of approximately 90 degrees from the application of the axial compression force.

7. The medical device of claim 6, wherein the second plurality of living hinges provides at least one of a turn and an arc of approximately 90 degrees from the application of the axial compression force.

8. The medical device of claim 7, wherein each of the third and fourth pluralities of living hinges provide at least one of a turn and an arc of approximately 90 degrees from the application of the axial compression force.

9. The medical device of claim 1, wherein the shaping structure extends along a substantial length of the medical device.

10. The medical device of claim 1, further comprising a pull wire coupled to the handle and the shaping structure, wherein the pull wire is configured to apply an axial compression force to at least a portion of the shaping structure.

11. The medical device of claim 10, wherein the shaping structure defines a lumen therethrough, the lumen defining an oblong cross-sectional opening, and wherein the pull wire traverses the lumen.

12. The medical device of claim 1, wherein the flexible conduit is configured to substantially resist axial compression.

13. The medical device of claim 1, further comprising a probe which communicates directly with a radiofrequency (RF) ablation catheter or a mapping catheter.

14. A medical device, comprising:
a handle;
a flexible conduit having a proximal segment and a distal segment, wherein the proximal segment is coupled to the handle, and wherein the flexible conduit includes at least one of a stainless steel hypotube and a nitinol hypotube; and
a substantially contiguous shaping structure coupled to the distal segment of the flexible conduit, wherein the shaping structure is configured to transition from (i) a substantially linear configuration to (ii) a configuration where a portion of the shaping structure is laterally displaced from remaining portions of the contiguous shaping structure upon the application of an axial compression force to the shaping structure.

15. A medical device, comprising:
a handle;
a flexible conduit having a proximal segment and a distal segment, wherein the proximal segment is coupled to the handle; and
a substantially contiguous shaping structure coupled to the distal segment of the flexible conduit, wherein the shaping structure is configured to transition from (i) a substantially linear configuration to (ii) a configuration where a portion of the shaping structure is laterally displaced from remaining portions of the contiguous shaping structure upon the application of an axial compression force to the shaping structure; and
at least one balloon coupled to the shaping structure.

16. The medical device of claim 15, wherein the at least one balloon is non-concentric with the shaping structure.

17. The medical device of claim 15, wherein the at least one balloon is expandable asymmetrically about a circumference of the shaping structure.

18. The medical device of claim 15, wherein the at least one balloon has at least one of a substantially semi-circular cross-section and a substantially flattened surface segment when inflated.

19. The medical device of claim 15, wherein the at least one balloon comprises a plurality of balloons, and wherein at least one of the plurality of balloons is radially offset with respect to at least one other balloon.

20. The medical device of claim 15, wherein the shaping structure comprises a first plurality of living hinges and a second plurality of living hinges.
